(19) United States
(12) Patent Application Publication
(10) Pub. No.: US 2007/0269891 A9
(43) Pub. Date: Nov. 22, 2007

(54) Solid surface with immobilized degradable cationic polymer for transfecting eukaryotic cells

(76) Inventors: Yasunobu Tanaka, San Diego, CA (US); Chris P. Castello, Vista, CA (US); Lei Yu, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/303,082

(22) Filed: Dec. 14, 2005

Related U.S. Application Data
(63) Continuation-in-part of application No. 10/341,059, filed on Jan. 13, 2003.
(60) Provisional application No. 60/637,344, filed on Dec. 17, 2004.

(51) Int. Cl. C12N 15/87; C12N 15/82; C12M 1/42

(52) U.S. Cl. 435/455; 435/468; 435/285.2

(57) ABSTRACT

A cell transfection/culture device is disclosed which includes a solid support coated with a degradable polymer cation as a transfection reagent. The transfection/culture device is conveniently stored at room temperature until use. Cell transfection is accomplished easily by adding the nucleic acid of interest and the cells to be transfected to the transfection/culture device. Cell transfection is completed in less than one hour by using the transfection/culture device described herein.

### RELATED APPLICATIONS

This application claims priority to U.S. provisional application No. 60/637,344, filed Dec. 17, 2004, which is incorporated herein by reference.

### BACKGROUND OF THE INVENTION

1. Field of the Invention

Embodiments of the invention relate to devices and methods for cell transfection. In particular, embodiments of the invention are directed to a cell transfection formula and to a cell culture device that has been treated with the transfection formula. The treated cell culture device can be stored at room temperature and provides a transfection method that is simple and quick.

2. Description of the Related Art

Gene transfection methods can be used to introduce nucleic acids into cells and are useful in studying gene regulation and function. High throughput assays that can be used to screen large sets of DNAs to identify those encoding products with properties of interest which are particularly useful. Gene transfection is the delivery and introduction of biologically functional nucleic acids into a cell, particularly a eukaryotic cell, in such a way that the nucleic acid retains its function within the cell. Gene transfection is widely applied in studies related to gene regulation, gene function, molecular therapy, signal transduction, drug screening, and gene therapy studies. As the cloning and cataloging of genes from higher organisms continues, researchers seek to discover the function of the genes and to identify gene products with desired properties. This growing collection of gene sequences requires the development of systematic and high-throughput approaches to characterizing gene products and analyzing gene function, as well as other areas of research in cell and molecular biology.

Both viral and non-viral gene carriers have been used in gene delivery. Viral vectors have been shown to have higher transfection efficiency than non-viral carriers, but the safety of viral vectors hampers applicability (Verma I. M and Somia N. Nature 389 (1997), pp. 239-242; Marhsall E. Science 286 (2000), pp. 2244-2245). Although non-viral transfection systems have not exhibited the efficiency of viral vectors, they have received significant attention, because of their theoretical safety when compared to viral vectors. In addition, viral vector preparation is a complicated and expensive process, which limits the application of viral vectors in vitro. The preparation of non-viral carriers is simpler and more cost effective in comparison to preparation of viral carriers, making synthetic gene carriers desirable as transfection reagents, particularly for in vitro studies.

Most non-viral vectors mimic important features of viral cell entry in order to overcome cellular barriers, which are meant to prevent infiltration by foreign genetic material. Non-viral gene vectors, based on a gene carrier backbone, can be classified as a) lipoplexes, b) polyplexes, and c) lipopolyplexes. Lipoplexes are assemblies of nucleic acids with a lipidic component, which is usually cationic. Gene transfer by lipoplexes is called lipofection. Polyplexes are complexes of nucleic acids with cationic polymer. Lipopolyplexes comprise both a lipid and a polymer component. Often such DNA complexes are further modified to contain a cell targeting or an intracellular targeting moiety and/or a membrane-destabilizing component, for example, a viral protein or peptide or a membrane-disruptive synthetic peptide. Recently, bacteria and phages have also been described as shuttles for the transfer of nucleic acids into cells.

Most non-viral transfection reagents are synthetic cationic molecules and have been reported to “coat” the nucleic acid by interaction of the cationic sites on the cation and anionic sites on the nucleic acid. The positively-charged DNA-cationic molecule complex interacts with the negatively charged cell membrane to facilitate the passage of the DNA through the cell membrane by non-specific endocytosis. (Schofield, Brit. Microencapsulated. Bull, 51(1):56-71 (1995)). In most conventional gene transfection protocols, the cells are seeded on cell culture devices 16 to 24 hours before transfection. The transfection reagent (such as a cationic polymer carrier) and DNA are usually prepared in separate tubes, and each respective solution is diluted in medium (containing no fetal bovine serum or antibiotics). The solutions are then mixed by carefully and slowing adding one solution to the other while continuously vortexing the mixture. The mixture is incubated at room temperature for 15-45 minutes to allow complex formation between the transfection reagent and the DNA and to remove residues of serum and antibiotics. Prior to transfection, the cell culture medium is removed and the cells are washed with buffer. The solution containing the DNA-transfection reagent complexes is added to the cells, and the cells are incubated for about 3-4 hours. The medium containing the transfection reagent is then be replaced with fresh medium. The cells are finally analyzed at one or more specific time point(s). This is obviously a time consuming procedure, particularly when the number of samples to be transfected is very large.

Several major problems exist in conventional transfection procedures. First, conventional procedures are time-consuming, particularly when there are many cell or gene samples to be used in transfection experiments. Also, the results derived from common transfection procedures are difficult to reproduce, due to the number of steps required. For instance, the DNA-transfection reagent complex formation is influenced by concentration and volume of nucleic acid and reagents, pH, temperature, type of buffer(s) used, length and speed of vortexing, incubation time, and other factors. Although the same reagents and procedure may be followed, different results may be obtained. Results derived from multi-step procedures are often influenced by human or mechanical error or other variations at each step. In addition, refreshing the cell culture medium following transfection disturbs the cells and may cause them to detach from the surface on which they are cultured, thus leading to variation and unpredictability in the final results. Due to all the factors noted, conventional transfection methods require a highly skilled individual to perform the transfection experiment or assay.

Researchers require an easier and more cost effective method of transfecting cells, and a high-throughput method of transfecting cells is needed in order to transfect large sample numbers efficiently.

Sabatini (U.S. 2002/0006664A1) describes a composition containing DNA which is deposited on a glass slide. However the system only allows transfection with the previously deposited DNA. This is a major disadvantage of this system. As it only provides for transfecting with previously deposited DNA, every researcher cannot use his or her desired nucleic acids.

U.S. Publication No. 2004/0138154A1, which is incorporated herein by reference, describes a cell culture/transfection device where the transfection is mediated by a lipid polymer. U.S. Publication No. 2005/0176132A1, also incorporated herein by reference, describes a calcium salt mediated transfectable cell culture device.

U.S. Publication No. 2003/0215395A1, incorporated herein by reference, describes degradable polymers which can be used for gene delivery.

As discussed above, conventional transfection is a lengthy and technically difficult procedure. Generally, three steps are required: 1) cells are seeded in a cell culture plate or dish and incubated until sufficient confluence is achieved; 2) transfection reagent/nucleic acid complexes are prepared; and 3) nucleic acids of interest are added along with the transfection reagent and further incubation is carried out. Two incubation periods are needed and typically it takes more than two days to complete all the steps. In contrast, embodiments of the present invention provide a simple procedure that involves only a single incubation step. A cell culture device, which has previously been coated with a transfection reagent, allows transfection by adding the nucleic acid of interest and the cell culture in succession. The transfected cells may then be cultured in the same device. Thus the cells may be transfected and cultured in the cell culture device without the need for further manipulation of the cells immediately after the transfection step. Transfection efficiency is comparable to regular transfection, but the time required for the operation is reduced by more than one day. Embodiments of the invention include a transfectable cell culture device which greatly reduces the labor of transfection assays, and enables transfection with any nucleic acid of interest in an easy method with low cytotoxicity. Also, the transfectable cell culture device of the invention is stable for long term storage at room temperature.

### SUMMARY OF THE INVENTION

Embodiments of the invention are directed to a device which includes a solid support coated with a transfection reagent mixture. Preferably, the transfection reagent in the coating is not complexed with a biomolecule, such as a nucleic acid. Preferably, the solid support is polystyrene resin, epoxy resin or glass. Preferably, the coating is on the surface of the solid support. Preferably, the coating amount of the transfection reagent is from about 0.1 to about 100 μg/cm2. Preferably, the transfection agent is a polymer. More preferably, the polymer is a cationic polymer. Preferably, the transfection agent comprises a degradable cationic polymer. More preferably, the degradable cationic polymer is made by linking cationic compounds or oligomers with degradable linkers. The transfection agent may comprise both a degradable cationic polymer and a non-degradable cationic polymer. Preferably, the ratio of the non-degradable cationic polymer to the degradable cationic polymer is 1:0.5 to 1:20 (non-degradable:degradable) by weight.

In preferred embodiments, the transfection reagent includes a plurality of cationic molecules and at least one degradable linker molecule connecting said cationic molecules in a branched arrangement, wherein said cationic molecules are selected from:
(i) a cationic compound of formula (A) or (B) or a combination thereof: 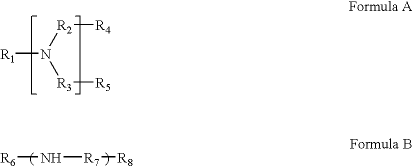
   wherein R¹ is a hydrogen atom, an alkyl of 2 to 10 carbon atoms, another Formula A, or Formula B;
R² is a straight chain alkylene group of the formula: —(CH₂)ₐ— wherein a is an integer number from 2 to 10;
R³ is a straight or branched chain alkylene group of the formula: —(C_{b}H_{2b})— wherein b is an integer number from 2 to 10;
R⁴ is a hydrogen atom, an alkyl of 2 to 10 carbon atoms, another Formula A, or Formula B;
R⁵ is a hydrogen atom, an alkyl of 2 to 10 carbon atoms, another Formula A, or Formula B;
R⁶ is a hydrogen atom, an alkyl of 2 to 10 carbon atoms, Formula A, or another Formula B;
R⁷ is a straight or branched chain alkylene group of the formula: —(C_{c}H_{2c})— in which c is an integer number from 2 to 10; and
R⁸ is a hydrogen atom, an alkyl of 2 to 10 carbon atoms, Formula A, or another Formula B;
(ii) a cationic dendritic or branched polyamidoamine (PAMAM) with terminated primary or secondary amino groups;
(iii) a cationic polyamino acid; or
(iv) a cationic polycarbohydrate;
   and wherein said degradable linker molecule is represented by the formula:
   A(Z)_{d}
   wherein A is a spacer molecule having at least one degradable bond, Z is a reactive residue which reacts with amino group, and d is an integer equal to or more than two and wherein A and Z are bound covalently.

In preferred embodiments, the cationic compound or oligomer is poly(L-lysine) (PLL), polyethyleneimine (PEI), polypropyleneimine (PPI), pentaethyleneamine, N,N′-bis(2-aminoethyl)-1,3-propanediamine, N,N′-bis(2-aminopropyl)-ethylenediamine, spermine, spermidine, N-(2-aminoethyl)-1,3-propanediamine, N-(3-aminopropyl)-1,3-propanediamine, tri(2-aminoethyl)amine, 1,4-bis(3-aminopropyl)piperazine, N-(2-aminoethyl)piperazine, dendritic polyamidoamine (PAMAM), chitosan, or poly(2-dimethylamino)ethyl methacrylate (PDMAEMA).

In preferred embodiments, the linker molecule is di- and multi-acrylates, di- and multi-acrylamides, di- and multi-isothiocyanates, di- and multi-isocyanates, di- and multi-epoxides, di- and multi-aldehydes, di-and multi-acyl chlorides, di- and multi-sulfonyl chlorides, di- and multi-halides, di- and multi-anhydrides, di- and multi-maleimides, di- and multi-N-hydroxysuccinimide esters, di- and multi-carboxylic acids, or di-and multi-a-haloacetyl groups.

In preferred embodiments, the linker molecule is 1,3-butanediol diacrylate, 1,4-butanediol diacrylate, 1,6-hexanediol diacrylate, 2,4-pentanediol diacrylate, 2-methyl-2,4-pentanediol diacrylate, 2,5-dimethyl-2,5-hexanediol diacrylate, poly(ethylene glycol) diacrylate, trimethylolpropane triacrylate, pentaerythritol tetraacrylate, di(trimethylolpropane) tetraacrylate, dipentaerythritol pentaacrylate, or a polyester with at least three acrylate or acrylamide side groups.

In preferred embodiments, the molecular weight of the polymer is from 500 da to 1,000,000 da. More preferably, the molecular weight of the polymer is from 2000 da to 200,000 da.

In preferred embodiments, the molecular weight of the cationic compound or oligomer is from 50 da to 10,000 da. In preferred embodiments, the molecular weight of the linker molecule is from 100 da to 40,000 da.

Preferably, the solid support is a dish bottom, a multi-well plate, or a continuous surface.

In some preferred embodiments, the transfection agent is covalently associated with a nucleic acid. In other preferred embodiments, the transfection agent is non-covalently associated with a nucleic acid.

In preferred embodiments, the device can be stored at room temperature for at least 5 months without significant loss of transfection activity.

Embodiments of the invention are directed to a method of cell transfection which includes the steps of adding a solution including a nucleic acid to be transfected to a device which includes a solid support coated with a transfection reagent mixture, adding eukaryotic cells to the solution; and incubating the cells and the nucleic acid solution to allow cell transfection. Preferably, the incubation is for 5 min. to 3 hours. More preferably, the incubation is for 10 min. to 90 min.

Preferably, the nucleic acid is DNA, RNA, DNA/RNA hybrid or chemically-modified nucleic acid. More preferably, the DNA is circular (plasmid), linear, fragment or single strand oligonucleotide (ODN). More preferably, the RNA is single strand (ribozyme) or double strand (siRNA).

In some preferred embodiments, the cell is a mammalian cell. In some preferred embodiments, at least some of the cells undergo cell division. In some preferred embodiments, the cell is a transformed or primary cell. In some preferred embodiments, the cell is a somatic or stem cell. In some preferred embodiments, the cell is a plant cell.

Further aspects, features and advantages of this invention will become apparent from the detailed description of the preferred embodiments which follow.

### BRIEF DESCRIPTION OF THE DRAWINGS

These and other features of this invention will now be described with reference to the drawings of preferred embodiments which are intended to illustrate and not to limit the invention.

FIG. 1 shows the cell shape of transfected 293 cells. The transfection agent treatments were linear polyethyleneimine (L-PEI) based polymer, lipid based polymer, degradable cationic polymer, and no treatment (intact 293 cells).

FIG. 2 shows percentage of EGFP-positive cells.

FIG. 3 shows cell condition after transfection.

FIG. 4 shows the stability of a transfectable cell culture device in a mylar bag with O₂ absorber.

FIG. 5 shows the stability of a transfectable cell culture device in a mylar bag with CO₂ absorber.

FIG. 6 shows the stability of a transfectable cell culture device in a mylar bag with O₂ and CO₂ absorber.

FIG. 7 shows the stability of a transfectable cell culture device in a mylar bag.

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

Embodiments of the invention are directed to a transfection device and method which is simple, convenient, and efficient compared to conventional transfection assays. A transfection device is made according to methods described herein by affixing a transfection reagent on the solid surface of a cell culture device. By using this device, researchers need only add a nucleic acid or other biomolecule to be transfected and cells to the surface of the cell culture device. There is no need to pre-mix the DNA or biomolecule with a transfection reagent. This removes a key timing-consuming step, which is required by conventional transfection procedures. Only approximately 40 minutes is required to complete the entire transfection process for 10 samples, compared to 2 to 5 hours or more required by current methods. This is particularly advantageous for high throughput transfection assays, in which hundreds of samples are tested at a time.

As compared to conventional transfection, there are several advantages to the method described herein. It provides a transfection device that is very easy to store, and it provides a simple method for biomolecule delivery or gene transfection in which no biomaterial/transfection reagent mixing step is required. The transfection procedure described herein can be finished in a short period of time, for instance approximately 5 min. to 3 hours, and it provides a high throughput method for transfection or drug delivery in which large numbers of samples may be transfected at a time.

In preferred embodiments, transfection reagents are simply coated onto the surface of a cell culture device, which can be easily commercialized and mass-produced. Customers, researchers for instance, need only add a biomolecule, such as a nucleic acid of interest, directly to the surface of a cell culture device in order to prepare the device prior to addition of cells. An incubation period for a predetermined time allows the biomolecule and the transfection reagent(s) to form a complex for uptake by cells in the next step. Cells are then seeded on the surface of the cell culture device and incubated, without the necessity of changing the medium, and the cells are analyzed. Changing medium during the transfection procedure is unnecessary. The methods described herein dramatically reduce the risk of error, by reducing the number of steps involved, thus increasing consistency and accuracy of the system.

The composition containing the transfection agent can be affixed to any suitable surface. For example, the surface can be glass, plastics (such as polytetrafluoroethylene, polyvinylidenedifluoride, polystyrene, polycarbonate, polypropylene), silicon, metal (such as gold), membranes (such as nitrocellulose, methylcellulose, PTFE or cellulose), paper, biomaterials (such as protein, gelatin, agar), tissues (such as skin, endothelial tissue, bone, cartilage), or minerals (such as hydroxylapatite, graphite). According to preferred embodiments the surfaces may be slides (glass or poly-L-lysine coated slides) or wells of a multi-well plate.

For slides, such as a glass slide coated with poly-L-lysine (e.g., Sigma, Inc.), the transfection reagent is fixed on the surface and dried, and then a nucleic acid of interest or a nucleic acid to be introduced into cells is introduced. Generally, the nucleic acid is spotted onto the glass slide in a microarray. The slide is incubated at room temperature for 30 minutes to form nucleic acid/transfection reagent complexes on the surface of the transfection device. The nucleic acid/transfection reagent complexes create a medium for use in high throughput microarrays, which can be used to study hundreds to thousands of nucleic acids, or other biomolecules at the same time. In an alternative embodiment, the transfection reagents can be affixed on the surface of the transfection device in discrete, defined regions to form a microarray of transfection reagents. In this embodiment, biomolecules, such as nucleic acids, which are to be introduced into cells, are spread on the surface of the transfection device and incubated with the transfection reagent microarray. This method can be used in screening transfection reagents or other delivery reagents from thousands of compounds. The results of such a screening method can be examined through computer analysis.

In another embodiment of the invention one or more wells of a multi-well plate may be coated with one or more transfection reagent(s). Plates commonly used in transfection are 96-well and 384-well plates. The transfection reagent can be evenly applied to the bottom of each well in the multi-well plate. Generally, the transfection reagent is applied to the bottom of plate in the range of about 0.1 to about 100 μg/cm². Further, the coating amount of the transfection reagent may be varied depending on the type of well plate to be used. For example, for a 6-well plate, 12-well plate or 96-well plate, the coating concentration of the transfection reagent is preferably from about 0.5 to about 50 μg/cm², and more preferably from about 1 to 20 μg/cm². In the case of a 384-well plate, the coating concentration of the transfection reagent is preferably from about 0.5 to about 50 μg/cm², and more preferably from about 1 to 30 μg/cm². In another embodiment of the invention, a 10-cm cell culture dish is coated with a transfection reagent. The transfection reagent can be evenly applied to the bottom of dish. The transfection reagent may be applied to the bottom of dish in the range of about 0.1 to about 100 μg/cm², more preferably about 0.2 to about 20 μg/cm².

Hundreds of nucleic acids or other biomolecules are then added into the well(s) by, for instance, a multichannel pipette or automated machine. Results of transfection are then determined by using a microplate reader. This is a very convenient method of analyzing the transfected cells, because microplate readers are commonly used in most biomedical laboratories. The multi-well plate coated with transfection reagent can be widely used in most laboratories to study gene regulation, gene function, molecular therapy, and signal transduction. Also, if different kinds of transfection reagents are coated on the different wells of multi-well plates, the plates can be used to screen many transfection or delivery reagents efficiently. Recently, 1,536 and 3,456 well plates have been developed, which may also be used according to the methods described herein.

In preferred embodiments, the transfection device is stored in a material suitable for packaging which may be plastic (e.g., cellophane), an elastomeric material, thin metal, Mylar®, or other polyester film material. The storage may be with or without oxygen and/or carbon dioxide absorbers. The transfection plates prepared as described herein may be stored for at least 5 months at room temperature with retention of significant cell-transfecting activity.

The transfection reagent is preferably a cationic compound which can introduce biomolecules, such as nucleic acids into cells. Preferred embodiments use cationic oligomers, such as low molecular weight polyethyleneimine (PEI). More preferably, the transfection agent is a degradable cationic polymer. Optionally, the transfection agent includes a cell-targeting or an intracellular-targeting moiety and/or a membrane-destabilizing component, as well as delivery enhancers.

In general, delivery enhancers fall into two categories. These are viral carrier systems and non-viral carrier systems. As human viruses have evolved ways to overcome the barriers to transport into the nucleus discussed above, viruses or viral components are useful in transport of nucleic acid into cells. Additionally, the degradable polymers may be conjugated to or associated with a viral or non-viral protein to enhance transfection efficiency. For example, vesicular stomatitis virus G protein (VSVG) and other peptides or proteins which are known to those of skill in the art may be added to the polymers in order to improve transfection efficiency.

Another example of a viral component useful as a delivery enhancer is the hemagglutinin peptide (HA-peptide). This viral peptide facilitates transfer of biomolecules into cells by endosome disruption. At the acidic pH of the endosome, this protein causes release of the biomolecule and carrier into the cytosol.

Non-viral delivery enhancers may be either polymer-based or lipid-based. They are generally polycations which act to balance the negative charge of the nucleic acid. Polycationic polymers have shown significant promise as non-viral gene delivery enhancers due in part to their ability to condense DNA plasmids of unlimited size and to safety concerns with viral vectors. Examples include peptides with regions rich in basic amino acids such as oligo-lysine, oligo-arginine or a combination thereof and polyethylenimine (PEI). These polycationic polymers facilitate transport by condensation of DNA. Branched chain versions of polycations such as PEI and Starburst dendrimers can mediate both DNA condensation and endosome release (Boussif, et al. (1995) Proc. Natl. Acad. Sci USA vol. 92: 7297-7301). PEI is a highly branched polymer with terminal amines that are ionizable at pH 6.9 and internal amines that are ionizable at pH 3.9 and because of this organization, can generate a change in vesicle pH that leads to vesicle swelling and eventually, release from endosome entrapment.

Another means to enhance delivery is to design a ligand on the transfection reagent. The ligand must have a receptor on the cell that has been targeted. Biomolecule delivery into the cell is then initiated by receptor recognition. When the ligand binds to its specific cell receptor, endocytosis is stimulated. Examples of ligands which have been used with various cell types to enhance biomolecule transport are galactose, transferrin, the glycoprotein asialoorosomucoid, adenovirus fiber, malaria circumsporozite protein, epidermal growth factor, human papilloma virus capsid, fibroblast growth factor and folic acid. In the case of the folate receptor, the bound ligand is internalized through a process termed potocytosis, where the receptor binds the ligand, the surrounding membrane closes off from the cell surface, and the internalized material then passes through the vesicular membrane into the cytoplasm (Gottschalk, et al. (1994) Gene Ther 1:185-191).

Various agents have been used for endosome disruption. Besides the HA-protein described above, defective-virus particles have also been used as endosomolytic agents (Cotten, et al. (July 1992) Proc. Natl. Acad. Sci. USA vol. 89: pages 6094-6098). Non-viral agents are either amphiphillic or lipid-based.

The release of biomolecules such as DNA into the cytoplasm of the cell can be enhanced by agents that either mediate endosome disruption, decrease degradation, or bypass this process all together. Chloroquine, which raises the endosomal pH, has been used to decrease the degradation of endocytosed material by inhibiting lysosomal hydrolytic enzymes (Wagner, et al. (1990) Proc Natl Acad Sci USA vol. 87: 3410-3414). Branched chain polycations such as PEI and starburst dendrimers also promote endosome release as discussed above.

To completely bypass endosomal degradation, subunits of toxins such as Diptheria toxin and Pseudomonas exotoxin have been utilized as components of chimeric proteins that can be incorporated into a gene/gene carrier complex (Uherek, et al.(1998) J Biol. Chem. vol. 273: 8835-8841). These components promote shuttling of the nucleic acid through the endosomal membrane and back through the endoplasmic reticulum.

Once in the cytoplasm, the nucleic acid must find its way to the nucleus. Localization to the nucleus may be enhanced by inclusion of a nuclear localization signal on the nucleic acid-carrier. A specific amino acid sequence that functions as a nuclear-localization signal (NLS) is used. The NLS on a cargo-carrier complex interacts with a specific nuclear transport receptor protein located in the cytosol. Once the cargo-carrier complex is assembled, the receptor protein in the complex is thought to make multiple contacts with nucleoporins, thereby transporting the complex through a nuclear pore. After a cargo-carrier complex reaches its destination, it dissociates, freeing the cargo and other components.

Subsequences from the SV40 large T-antigen has been used for transport into nuclei. This short sequence from SV40 large T-antigen acts as a signal that causes the transport of associated macromolecules into the nucleus.

Biodegradable cationic polymers typically exhibit low cytotoxicity, but also low transfection efficiency due to rapid degradation, making them less competitive against other carriers for gene transfer and other applications. These degradable cationic polymers improve transfection efficiency by linking low molecular weight cationic compounds or oligomers together with degradable linkers. The linker molecules may contain biologically, physically or chemically cleavable bonds, such as hydrolysable bonds, reducible bonds, a peptide sequence with enzyme specific cleavage sites, pH sensitive, or sonic sensitive bonds. The degradation of these polymers may be achieved by methods including, but not limited to hydrolysis, enzyme digestion, and physical degradation methods, such as optical cleavage (photolysis).

One of the advantages of the degradable cationic polymers described herein is that degradation of the polymers is controllable in terms of rate and site of polymer degradation, based on the type and structures of the linkers.

In preferred embodiments, the transfection reagent includes a plurality of cationic molecules and at least one degradable linker molecule connecting said cationic molecules in a branched arrangement.

Cationic oligomers, such as low molecular weight polyethyleneimine (PEI), low molecular weight poly(L-lysine) (PLL), low molecular weight chitosan, and low molecular weight PAMAM dendrimers, can be used to make the polymers described herein. Furthermore, any molecule containing amines with more than three reactive sites can be used.

Cationic oligomers may be selected from, but are not limited to:
(i) a cationic compound of formula (A) or (B) or a combination thereof: 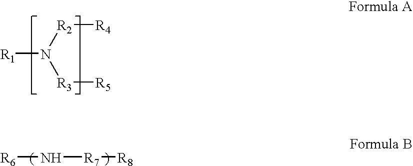
   wherein R₁ is a hydrogen atom, an alkyl of 2 to 10 carbon atoms, another Formula A, or Formula B;
R₂ is a straight chain alkylene group of the formula: —(CH₂)ₐ— wherein a is an integer number from 2 to 10;
R₃ is a straight chain alkylene group of the formula: —(C_{b}H_{2b})— wherein b is an integer number from 2 to 10;
R₄ is a hydrogen atom, an alkyl of 2 to 10 carbon atoms, another Formula A, or Formula B;
R₅ is a hydrogen atom, an alkyl of 2 to 10 carbon atoms, another Formula A, or Formula B;
R₆ is a hydrogen atom, an alkyl of 2 to 10 carbon atoms, Formula A, or another Formula B;
R₇ a straight or branched chain alkylene group of the formula: —(C_{c}H_{2c})— in which c is an integer number from 2 to 10; and
R₈ is a hydrogen atom, an alkyl of 2 to 10 carbon atoms, Formula A, or another Formula B;
(ii) a cationic dendritic or branched polyamidoamine (PAMAM) with terminated primary or secondary amino groups;
(iii) a cationic polyamino acid; and
(iv) a cationic polycarbohydrate.

Examples of such cationic molecules include, but are not limited to, the cationic molecules shown in Table 1.

TABLE 1
Structures of cationic compounds and oligomers according to preferred
embodiments of the invention

| Symbol | Name | Structure |
|---|---|---|
| C1 | Pentaethylenehexamine | 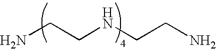 |
| C2 | Linear polyethylenimine Mw = 423) | 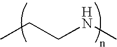 |
| C3 C4 | Branched polyethylenimine (Mw = 600) Branched polyethylenimine (Mw = 1200) | 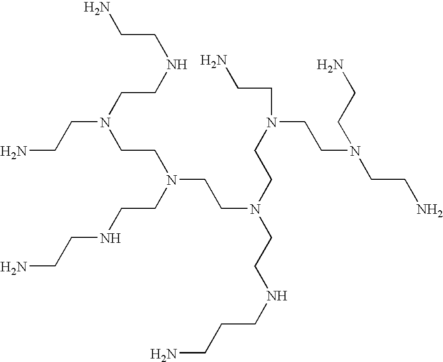 |
| C5 | N,N′-Bis(2-aminopropyl)- ethylenediamine | 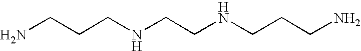 |
| C6 | Spermine | 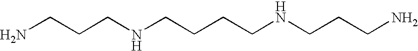 |
| C7 | N-(2-aminoethyl)-1,3- propanediamine | 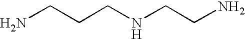 |
| C8 | N-(3-aminopropyl)-1,3- propanediamine | 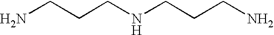 |
| C9 | N,N′-Bis(2-aminoethyl)- 1,3-propanediamine | 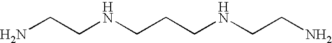 |
| C10 | Poly(amidoamine) | PAMAM |
| | Dendrimer | |
| C11 | Poly(propyleneimine) | DAB-Am-16 |
| | dendrimer | |
| C12 | Spermidine | 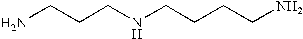 |
| C13 | 1,4-Bis(3-aminopropyl) piperazine | 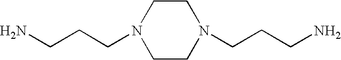 |
| C14 | 1-(2- Aminoethyl)piperazine | 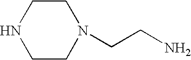 |
| C15 | Tri(2-aminoethyl)amine | 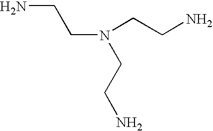 |
| C16 | Poly(L-lysine) | |

Cationic polymers used herein may include primary or secondary amino groups, which can be conjugated with active ligands, such as sugars, peptides, proteins, and other molecules. In a preferred embodiment, lactobionic acid is conjugated to the cationic polymers. The galactosyl unit provides a useful targeting molecule towards hepatocyte cells due to the presence of galactose receptors on the surface of the cells. In a further embodiment, lactose is conjugated to the degradable cationic polymers in order to introduce galactosyl units onto the polymer.

Degradable linking molecules include, but are not limited to, di- and multi-acrylates, di- and multi-methacrylates, di- and multi-acrylamides, di- and multi-isothiocyanates, di- and multi-isocyanates, di- and multi-epoxides, di- and multi-aldehydes, di- and multi-acyl chlorides, di- and multi-sulfonyl chlorides, di- and multi-halides, di- and multi-anhydrides, di- and multi-malemides, di- and multi-carboxylic acids, di- and multi-α-haloacetyl groups, and di- and multi-N-hydroxysuccinimide esters, which contain at least one biodegradable spacer. The following formula describes a linker which may be used according to preferred embodiments:
A(Z)_{d}
wherein A is a spacer molecule having at least one degradable bond, Z is a reactive residue which reacts with amino group, and d is an integer equal to or more than two and wherein A and Z are bound covalently.

Several embodiments of reactive residues of the linker molecules have been illustrated in Table 2, however these examples are not limiting to the scope of the invention. Reactive residues may be selected from, but are not limited to, acryloyl, maleimide, halide, carboxyl acylhalide, isocyanate, isothiocyanate, epoxide, aldehyde, sulfonyl chloride, and N-hydroxysuccinimide ester groups or combinations thereof.

TABLE 2
Structures of biodegradable linker molecules used in preferred
embodiments of the invention

| Symbol | Name | Structure |
|---|---|---|
| L1 | 1,3-Butanediol diacrylate | 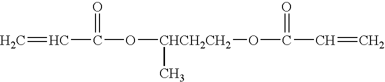 |
| L2 | 2-Methyl-2,4- pentanediol diacrylate | 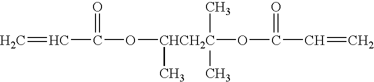 |
| L3 | Trimethylolpropane triacrylate | 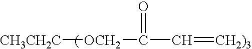 |
| L4 | 2,4-Pentanediol diacrylate | 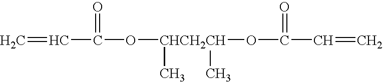 |
| L5 | Pentaerythritol tetraacrylate | 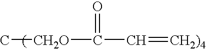 |
| L6 | Dipentaerythritol pentaacrylate | 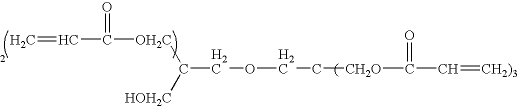 |
| L7 | Di(trimethylolpro- pane) tetraacrylate | 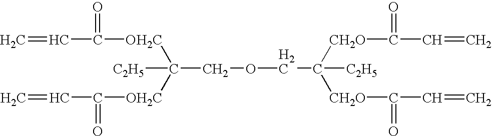 |
| L8 | 1,4-Butanediol diacrylate | 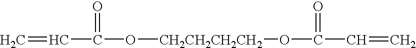 |
| L9 | 1,6-Hexanediol diacrylate | 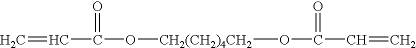 |
| L10 | 2,5-Dimethyl-2,5- hexanediol diacrylate | 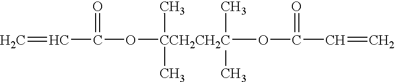 |

The degradation rates of the polymers can be controlled by changing the polymer composition, feed ratio, and the molecular weight of the polymers. For example, when linkers with bulkier alkyl groups are used, the polymers will degrade slower. Also, increasing molecular weight will cause a decrease in the degradation rate in some cases. Degradation rates of the polymers may be controlled by adjusting the ratio of cationic polymer to linker or by changing the various degradable linker molecules.

Acrylate linkers are much cheaper than disulfide-containing linkers, because synthesis of the disulfide-containing linkers is more difficult. Acrylate linkers can be hydrolysable in any water solution. Therefore a polymer containing acrylate linkers can be degraded in various environments as long as it contains water. Thus, polymers containing acrylate linkers have broad applications compared to disulfide-linker-containing polymers. In addition, the degradation rate of polymers with disulfide-linkers are usually the same, but the degradation rate of polymers synthesized with acrylate linkers can vary depending on the different acrylate linkers used.

In some embodiments, the transfection reagent can be mixed with a matrix, such as proteins, peptides, polysaccharides, or other polymers. The protein can be gelatin, collagen, bovine serum albumin or any other protein that can be used in affixing proteins to a surface. The polymers can be hydrogels, copolymers, non-degradable or biodegradable polymers and biocompatible materials. The polysaccharide can be any compound that can form a membrane and coat the delivery reagent, such as chitosan. Other reagents, such as cytotoxicity reductive reagents, cell binding reagents, cell growing reagents, cell stimulating reagents or cell inhibiting reagents and the compounds for culturing specific cells, can be also affixed to the transfection device along with the transfection or delivery reagent. The transfection agent may comprise both a degradable cationic polymer and a non-degradable cationic polymer. The ratio of the non-degradable cationic polymer to the degradable cationic polymer is preferably from 1:0.5 to 1:20 (non-degradable:degradable) by weight, and more preferably from 1:2 to 1:10 by weight.

According to another embodiment, a gelatin-transfection reagent mixture, comprising transfection reagent (e.g., lipid, polymer, lipid-polymer or membrane destabilizing peptide) and gelatin that is present in an appropriate solvent, such as water or double deionized water, may be affixed to the transfection device. In a further embodiment a cell culture reagent (e.g., fibronectin, collagen, salts, sugars, protein, or peptides) may also be present in the gelatin-transfection reagent mixture. The mixture is evenly spread onto a surface, such as a slide or multi-well plate, thus producing a transfection surface bearing the gelatin-transfection reagent mixture. In alternative embodiments, different transfection reagent-gelatin mixtures may also be spotted in discrete regions on the surface of the transfection device. The resulting product is allowed to dry completely under suitable conditions such that the gelatin-transfection reagent mixture is affixed at the site of application of the mixture. For example, the resulting product can be dried at specific temperatures or humidity or in a vacuum-dessicator.

The concentration of transfection reagent present in the mixture depends on the transfection efficiency and cytotoxicity of the reagent. Typically there is a balance between transfection efficiency and cytotoxicity. At concentrations in which a transfection reagent is most efficient, while keeping cytotoxicity at an acceptable level, the concentration of transfection reagent is at the optimal level. The concentration of transfection reagent will generally be in the range of about 1.0 μg/ml to about 1000 μg/ml. In preferred embodiments, the concentration is from about 10 μg/ml to about 600 μg/ml. Similarly, the concentration of gelatin or another matrix depends on the experiment or assay to be performed, but the concentration will generally be in the range of 0.01% to 0.5% (w/v) of the transfection reagent solution.

In preferred embodiments, the molecules to be introduced into cells are nucleic acids. The nucleic acid can be DNA, RNA, DNA/RNA hybrid, peptide nucleic acid (PNA), etc. If the DNA used is present in a vector, the vector can be of any type, such as a plasmid (e.g., plasmid carrying green fluorescence protein (GFP) gene and/or luciferase (luc) gene) or viral-based vector (e.g. pLXSN). The DNA can also be linear fragment with a promoter sequence (such as CMV promoter) at the 5′ end of the cDNA to be expressed and a poly A site at the 3′ end. These gene expression elements allow the cDNA of interest to be transiently expressed in mammalian cells. If the DNA is a single strand oligodeoxynucleotide (ODN), for example antisense ODN, it can be introduced into cells to regulate target gene expression. In embodiments using RNA the nucleic acid may be single stranded (antisense RNA and ribozyme) or double stranded (RNA interference, SiRNA). In most cases, the RNA is modified in order to increase the stability of RNA and improve its function in down regulation of gene expression. In peptide nucleic acid (PNA), the nucleic acid backbone is replaced by peptide, which makes the molecule more stable. The methods described herein can be used to introduce nucleic acids into cells for various purposes, for example molecular therapy, protein function studies, or molecule mechanism studies.

Under appropriate conditions, a nucleic acid solution is added into the transfection device, which has been coated with the transfection reagent, to form a nucleic acid transfection reagent complex. The nucleic acids are preferably dissolved in cell culture medium without fetal bovine serum and antibiotics, for example Dulbecco's Modified Eagles Medium (DMEM). However, any appropriate cell culture media may be used including, but not limited to, Minimum Essential Eagle, F-12 Kaighn's Modification medium, or RPMI 1640 medium. If the transfection reagent is evenly affixed on the slide, the nucleic acid solution can be spotted onto discrete locations on the slide. Alternatively, transfection reagents may be spotted on discreet locations on the slide, and the nucleic acid solution can simply be added to cover the whole surface of the transfection device. If the transfection reagent is affixed on the bottom of multi-well plates, the nucleic acid solution is simply added into different wells by multi-channel pipette, automated device, or other delivery methods which are well known in the art. The resulting product (transfection device coated with transfection reagent and desired nucleic acid) is incubated for approximately 5 min. to 60 min., more preferably, from 25-30 minutes at room temperature to form the nucleic acid/transfection reagent complex. In some embodiments, for example, if different nucleic acid samples are spotted on discrete locations of the slide, the DNA solution will be removed to produce a surface bearing the nucleic acid samples in complex with the transfection reagent. In other alternate embodiments, the nucleic acid solution can be kept on the surface. Secondly, cells in an appropriate medium, such as DMEM, and appropriate density are plated onto the surface. The resulting product (a surface bearing biomolecules and plated cells) is maintained under conditions that result in entry of the nucleic acids of interest into the plated cells. In alternate embodiments, the cells are mixed with the biomolecule or nucleic acid. The cell/biomolecule mixture is then added to the transfection device and incubated at room temperature.

Suitable cells for use according to the methods described herein include prokaryotes, yeast, or higher eukaryotic cells, including plant and animal cells, especially mammalian cells. In preferred embodiments, eukaryotic cells, such as mammalian cells (e.g., human, monkey, canine, feline, bovine, or murine cells), bacterial, insect or plant cells, are plated onto the transfection device, which is coated with transfection reagent and nucleic acids of interest, in sufficient density and under appropriate conditions for introduction/entry of the nucleic acids of interest into the eukaryotic cells and either expression of the DNA or interaction of the biomolecule with cellular components. In particular embodiments the cells may be selected from hematopoietic cells, neuronal cells, pancreatic cells, hepatic cells, chondrocytes, osteocytes, or myocytes. The cells can be fully differentiated cells or progenitor/stem cells.

In preferred embodiments, eukaryotic cells are grown in Dulbecco's Modified Eagles Medium (DMEM) containing 10% heat-inactivated fetal bovine serum (FBS) with L-glutamine and penicillin/streptomycin (pen/strep). It will be appreciated by those of skill in the art that certain cells should be cultured in a special medium, because some cells need special nutrition, such as growth factors and amino acids. Appropriate media for culture of particular cell types are known to those of skill in the art. The optimal density of cells depends on the cell types and the purpose of experiment. For example, a population of 70-80% confluent cells is preferred for gene transfection, but for oligonucleotide delivery the optimal condition is 30-50% confluent cells. For example, if 5×10⁴ 293 cells/well were seeded onto a 96 well plate, the cells would reach 90% confluency at 18-24 hours after cell seeding. For HeLa 705 cells, only 1×10⁴ cells/well are needed to reach a similar confluent percentage in a 96 well plate.

After the cells are seeded on the surface containing the nucleic acid samples/transfection reagent, the cells are incubated under optimal conditions for the cell type (e.g. 37° C., 5-10% CO₂). The culture time is dependent on the purpose of experiment. Typically, the cells are incubated for 24 to 48 hours for cells to express the target gene in the case of gene transfection experiments. In the analysis of intracellular trafficking of biomolecules in cells, minutes to several hours of incubation may be required and the cells can be observed at defined time points.

The results of biomolecule delivery can be analyzed by different methods. In the case of gene transfection and antisense nucleic acid delivery, the target gene expression level can be detected by reporter genes, such as green fluorescent protein (GFP) gene, luciferase gene, or β-galactosidase gene expression. The signal of GFP can be directly observed under a microscope, the activity of luciferase can be detected by a luminometer, and the blue product catalyzed by β-galactosidase can be observed under a microscope or determined by a microplate reader. One of skill in the art is familiar with how these reporters function and how they may be introduced into a gene delivery system. The nucleic acid and its product, or other biomolecules delivered according to methods described herein and the target modulated by these biomolecules can be determined by various methods, such as detecting immunofluorescence or enzyme immunocytochemistry, autoradiography, or in situ hybridization. If immunofluorescence is used to detect expression of an encoded protein, a fluorescently labeled antibody that binds the target protein is used (e.g., added to the slide under conditions suitable for binding of the antibody to the protein). Cells containing the protein are then identified by detecting a fluorescent signal. If the delivered molecules can modulate gene expression, the target gene expression level can also be determined by methods such as autoradiography, in situ hybridization, and in situ PCR. However, the identification method depends on the properties of the delivered biomolecules, their expression product, the target modulated by it, and/or the final product resulting from delivery of the biomolecules.

### EXAMPLE 1

### Preparation of Degradable Cationic Polymer

The synthesis of a polymer which is derived from polyethylenimine oligomer with molecular weight of 600 (PEI-600) and 2,4-pentandiol diacrylate (PDODA) is provided as a general procedure for preparation of a degradable cationic polymer. To a vial, 4.32 g of PEI-600 in 25 ml of methylene chloride were added by using pipette or syringe. 2.09 g of PDODA was quickly added to the above PEI-600 solution with stirring. The reaction mixture was stirred for 4 hours at room temperature (20° C.). Then, the reaction mixture was neutralized by adding 50 ml of 2M HCl. The white precipitate was centrifuged, washed with methylene chloride, and dried at room temperature under reduced pressure.

### EXAMPLE 2

### Preparation of Transfectable Cell Culture Device with Degradable Cationic Polymer

Degradable cationic polymer was prepared as indicated in Example 1. Linear polyethyleneimine (L-PEI) based polymer and lipid based polymers were used for transfecting plasmid DNA into mammalian cells in vitro to evaluate the transfection efficiency. For L-PEI based polymer, jet PEI (Qbiogene) transfection reagent was used. Lipofectamine2000 (Invitrogen) and N-[1-(2,3-dioleoyloxy)propyl]-N,N,N-trimethylammonium salts (DOTAP; Sigma-Aldrich) were employed as lipid based polymers. Degradable cationic polymer and DOTAP were dissolved in methanol, and jet PEI and Lipofectamine2000 were diluted by deionized water. Flat bottom 96-well cell culture plates (bottom surface: 0.32 cm² per each well; BD Biosciences) were treated with these polymer solutions. The actual amounts affixed on the bottom were as follows: (a) Degradable cationic polymer; 3 μg per well, thus 9.4 μg/cm², (b) jet PEI; 1 μl per well, (c) Lipofectamine2000; 0.375 μg per well, (d) DOTAP; 2 and 4 pmole per well. These plates were dried at room temperature under reduced pressure and sealed in a vacuum pack until use.

### EXAMPLE 3

### Transfection with Transfectable Cell Culture Device for 293 Cells

25 or 50 ng of pEGFP-N1 plasmid (purchased from Clontech) in 25 μl of opti-MEM I (Invitrogen) was added in each well and kept at room temperature for 25 minutes. Then, 5×10⁴ of 293 cells in 100 μl of Dulbecco's modified Eagle Medium (DMEM) (Invitrogen) with 10% calf serum (Invitrogen) were added and incubated at 37° C. in 7.5% of CO₂. After 24 to 36 hrs. incubation, transfection efficiency was estimated by observing EGFP fluorescence by using epifluorescent microscope (IX70, Olympus).

Transfection efficiencies are shown in Table 3. Degradable cationic polymer and jet PEI, i.e. L-PEI based polymer showed high transfection efficiency.

TABLE 3

| Polymer | EGFP-positive cells |
|---|---|
| Degradable cationic polymer | 60-70% |
| Jet PEI | 50% |
| Lipofectamine2000 | Less than 10% |
| DOTAP 4 pmole/well | 0% |
| DOTAP 2 pmole/well | 0% |

### EXAMPLE 4

### Evaluation of Cytotoxicity

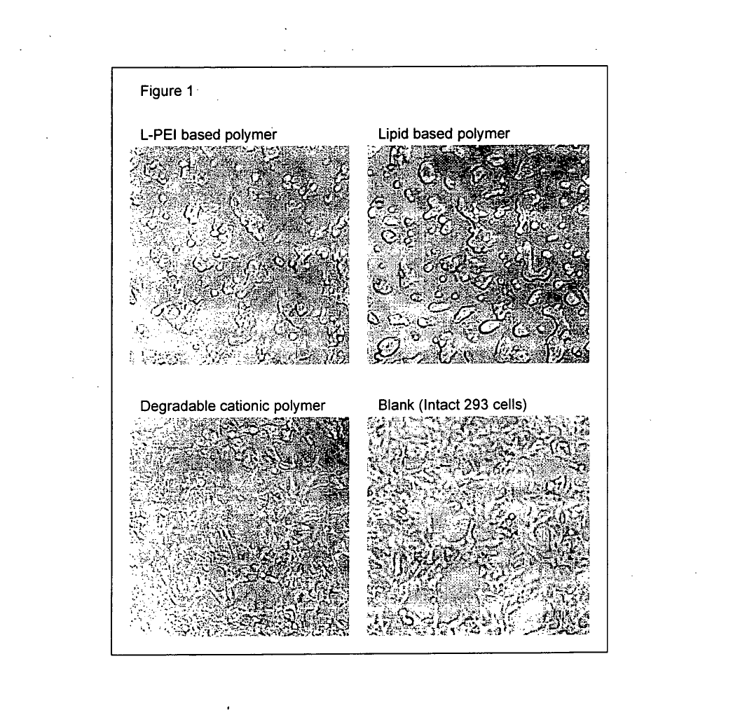

Cytotoxicity of the described method was evaluated. Cell shape of 293 cells, transfected as indicated in Example 3, were compared by microscopic observation (FIG. 1). Cells transfected by using degradable polymer showed normal shape, which was similar to intact 293 cells. However, those transfected by using L-PEI based polymer (jet PEI) and lipid based polymer (Lipofectamine2000) were rounded. We concluded that the degradable cationic polymer can deliver genes without damaging cells.

### EXAMPLE 5

### Optimization of Degradable Cationic Polymer Amount

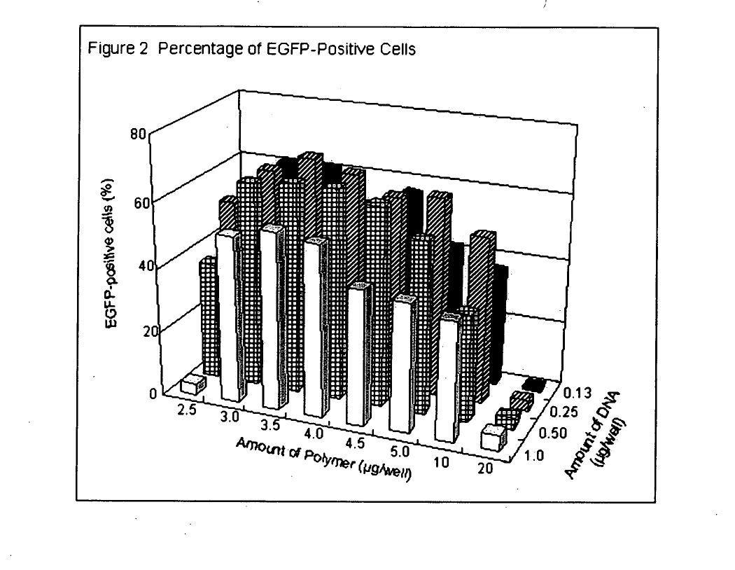

Various amounts of degradable cationic polymer were affixed on the cell culture devices, and transfection efficiency was evaluated. 96-well cell culture plates were coated with degradable cationic polymer by the same protocol as shown in Example 2. The actual amount of polymer was as follows: 2.5, 3.0, 3.5, 4.0, 4.5, 5.0, 10 and 20 μg per well. Then, transfection was carried out as described in Example 3 and transfected cells were incubated at 37° C. in 7.5% of CO₂. Amount of plasmid DNA added before seeding cells was 0.13, 0.25, 0.50 or 1.0 μg per well. After 40 hours incubation, percentage of fluorescing cells and cell condition were estimated by epifluorescent microscopy. FIG. 2 shows percentages of EGFP-positive cells after transfection. High transfection efficiencies were allocated between 2.5 to 5.0 μg per well (thus, 7.8 to 16 μg/cm²) of degradable cationic polymer with 0.25 and 0.5 μg per well (thus, 0.78 to 1.6 μg/cm²) of plasmid DNA.

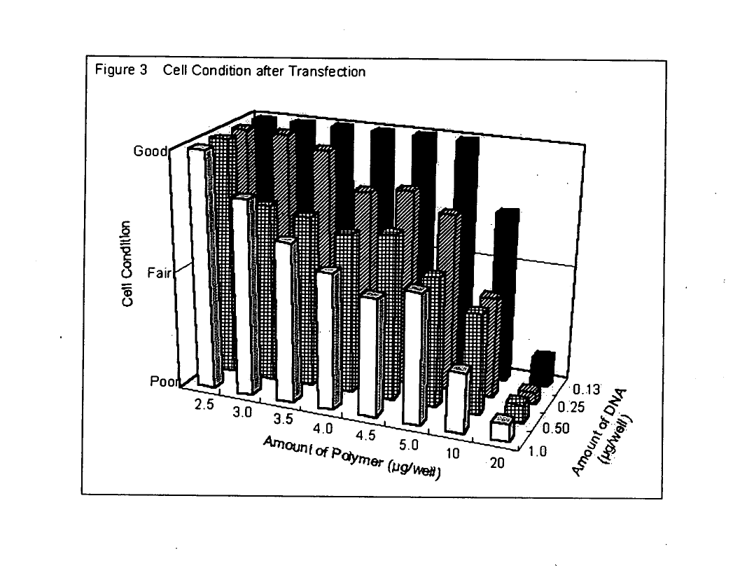

Cell condition in these experiments is shown in FIG. 3. Cells transfected in the plates with L-PEI and lipid based polymers had rounded shape and had aggregated. The morphology was due to cytotoxicity. Cell condition was acceptable when the amount of degradable cationic polymer affixed on the bottom of the plate was from 2.5 to 5.0 μg per well. Also, all the plasmid DNA conditions that we tested gave us good cell condition with degradable cationic polymer if the amount was from 2.5 to 5.0 μg per well.

### EXAMPLE 6

### Stability Study

There are products in the market, in which there is a coating on the surface of cell culture devices for a special purpose, for example, to assist cell growth. Normally, the coating material is a kind of protein, like collagen or fibronectin. As they are temperature-sensitive, these cell culture devices require refrigerated storage which is a disadvantage, especially if they are bulky. For this reason, stability at room temperature is an important feature.

The cell culture/transfection devices of this invention were tested to study their stability after long-term storage. The cell transfection devices were prepared as described in Example 2, and vacuum-sealed in Mylar Bags (Dupont Corp.), which is a film with an oxygen barrier material and aluminum foil with or without oxygen and carbon dioxide absorbers. Storage was at 25° C. Then, transfection efficiency with plasmid DNA carrying luciferase gene (pCMV-LUC) was tested periodically. The procedure for transfectable cell culture devices was as described in Example 3 except the plasmid DNA was different. Luciferase activity of cells were determined by using a Dynex MLX Microtiter® plate luminometer and Luciferase Assay System (Promega Corp. Madison, Wis. USA) to determine transfection efficiency.

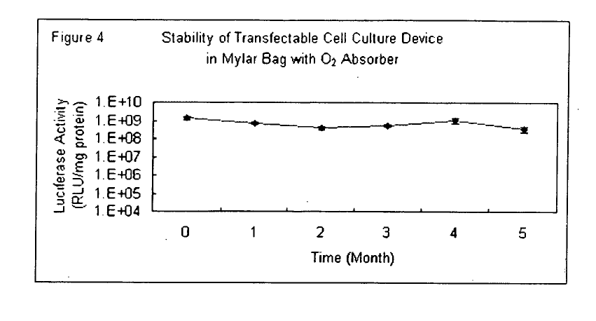

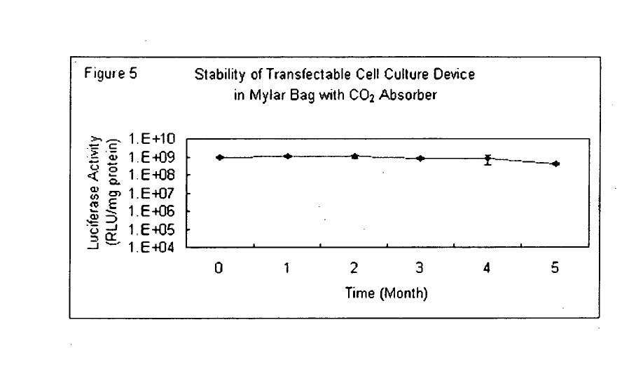

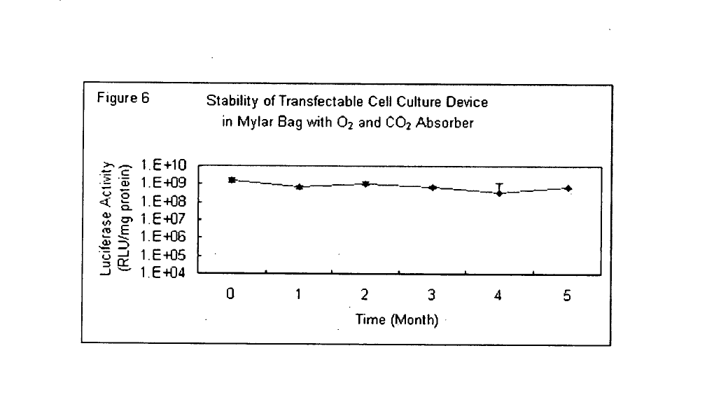

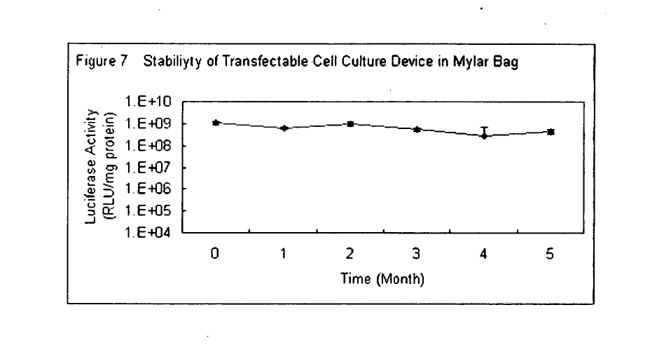

FIGS. 4, 5 and 6 show change of transfection efficiencies after storage at 25° C. with O₂ and/or CO₂ absorbing materials in Mylar Bags. There was no obvious decrease of transfection efficiency after 5 month storage. Moreover, even when cell culture devices were kept at 25° C. in Mylar Bags without O₂ and/or CO₂ absorbing materials, transfection efficiency was stable after 5 month and still quite high (FIG. 7). The cell culture devices of this invention are quite stable at room temperature. The device can be stored without special storage conditions.

### EXAMPLE 7

### Preparation of Non-Degradable Cationic Polymer

Non-degradable polymer was prepared as follows: Approximately 5 g of polyethlenimine (Aldrich, product number: 408727) was dissolved in 50 ml of dichloromethane, then 100 ml of 2.0M hydrogen chloride in diethyl ether (Aldrich, product number: 455180) was added and mixed well to form polymer hydrochloride. Then, the polymer hydrochloride was collected by centrifuge, and rinsed with 150 ml of diethyl ether. This rinse with diethyl ether was carried out twice. The resultant precipitation after the rinse was dried under vacuum condition at room temperature for 3 hours. Then, the dried powder was stored at 4° C. with desiccant until use.

### EXAMPLE 8

### Preparation of 96-Well Transfectable Cell Culture Device with Degradable Cationic Polymer and Non-Degradable Cationic Polymer

Degradable cationic polymer was prepared as indicated in Example 1. Non-degradable cationic polymer was obtained as described in Example 7. Both polymers were dissolved in methanol and mixed together to make a coating solution. The final concentration of each polymer was: Degradable cationic polymer; 40 μg/ml, and Non-degradable cationic polymer; 10 μg/ml. Then, flat bottom 96-well cell culture plates (bottom surface: 0.32 cm² per each well; BD Biosciences) were treated with the coating solution. Actually, 25 μl of the coating solution was dispensed in each well, and dried under vacuum condition to remove methanol. Under these coating conditions, 1 μg of degradable cationic polymer was affixed on each well of a 96-well plate; therefore the density of the degradable cationic polymer was 3.1 μg/cm². Also, 0.25 μg of non-degradable cationic polymer was affixed on each well of the 96-well plate so that the density of the non-degradable cationic polymer was 0.78 μg/cm². In total, 1.25 μg of polymer was affixed on each well of the 96-well plate; therefore the density of polymer was 3.9 μg/cm². The cell culture devices prepared in this example were vacuum sealed in Mylar Bags with desiccant, and stored at room temperature until further use.

### EXAMPLE 9

### Preparation of 12-Well Transfectable Cell Culture Device with Degradable Cationic Polymer and Non-Degradable Cationic Polymer

Degradable cationic polymer was prepared as indicated in Example 1. Non-degradable cationic polymer was obtained as described in Example 7. Both polymers were dissolved in methanol and mixed together to make a coating solution. The final concentration of each polymer was: Degradable cationic polymer; 80 μg/ml, and Non-degradable cationic polymer; 10 μg/ml. Then, flat bottom 12-well cell culture plates (bottom surface: 3.8 cm² per each well; BD Biosciences) were treated with these polymer solutions. 100 μl of the coating solution was dispensed in each well, and dried under vacuum condition to remove methanol. Under these coating conditions, 8.0 μg of degradable cationic polymer was affixed on each well of a 12-well plate so that the density of the degradable cationic polymer was 2.1 μg/cm²and 1.0 μg of non-degradable cationic polymer was affixed on each well of the 12-well plate so that the density of the non-degradable cationic polymer was 0.26 μg/cm². In total, 9.0 μg of polymer was affixed on each well of the 12-well plate; therefore the density of polymer was 2.4 μg/cm². The cell culture devices prepared in this example were vacuum sealed in Mylar Bags with desiccant, and stored at room temperature until further use.

### EXAMPLE 10

### Preparation of 6-Well Transfectable Cell Culture Device with Degradable Cationic Polymer and Non-Degradable Cationic Polymer

Degradable cationic polymer was prepared as indicated in Example 1. Non-degradable cationic polymer was obtained as described in Example 7. Both polymers were dissolved in methanol and mixed together to make a coating solution. The final concentration of each polymer was: Degradable cationic polymer; 80 μg/ml, and Non-degradable cationic polymer; 10 μg/ml. Then, flat bottom 6-well cell culture plates (bottom surface: 9.6 cm2 per each well; BD Biosciences) were treated with the coating solution. 200 μl of the coating solution was dispensed in each well, and dried under vacuum condition to remove methanol. Under these coating conditions, 16 μg of degradable cationic polymer was affixed on each well of a 6-well plate so that the density of the degradable cationic polymer was 1.7 μg/cm² and, 2.0 μg of non-degradable cationic polymer was affixed on each well of the 6-well plate so that the density of the non-degradable cationic polymer was 0.21 μg/cm². In total 18 μg of polymer was affixed on each well of the 6-well plate; therefore the density of polymer was 1.9 μg/cm². The cell culture devices prepared in this example were vacuum sealed in Mylar Bags with desiccant, and stored at room temperature until further use.

### EXAMPLE 11

### Transfection with 96-Well Transfectable Cell Culture Devices Prepared with Degradable and Non-Degradable Cationic Polymers

Mammalian cells were incubated in 10-cm cell culture dishes, rinsed with phosphate-buffered saline, and treated with trypsin solution. Then, the trypsinized cells were diluted in appropriate cell culture medium with serum to prepare a cell suspension. The cell density used in this example is shown in Table 4.

pEGFP-N1 plasmid was diluted in opti-MEM, and the final concentration was adjusted to 10 μg/ml. Then, 25 μl of the plasmid solution was added in each well of the 96-well transfectable cell culture device prepared as indicated in Example 8, and kept at room temperature for 25 minutes. Then, 100 μl of the cell suspension was added in the well, and incubated at 37° C. in 7.5% of CO₂. After 36 to 48-hour incubation, transfection efficiency was estimated by observing EGFP fluorescence by using epifluorescent microscope (IX70, Olympus).

Table 4 indicates the percentage of the cells with EGFP fluorescence in various mammalian cell lines. The 96-well transfectable cell culture device in this invention transfected various mammalian cell lines with high efficiency.

### EXAMPLE 12

### Transfection with 12-Well Transfectable Cell Culture Devices Prepared with Degradable and Non-Degradable Cationic Polymers

Mammalian cells were incubated in 10-cm cell culture dishes, rinsed with phosphate-buffered saline, and treated with trypsin solution. Then, the trypsinized cells were diluted in appropriate cell culture medium with serum to prepare cell suspension. The cell density used in this example is shown in Table 4.

pEGFP-N1 plasmid was diluted in opti-MEM, and the final concentration was adjusted to 5 μg/ml. Then, 200 μl of the plasmid solution was added in each well of the 12-well transfectable cell culture device prepared as indicated in Example 9, and kept at room temperature for 25 minutes. Then, 1 ml of the cell suspension was added in the well, and incubated at 37° C. in 7.5% of CO₂. After 36 to 48-hour incubation, transfection efficiency was estimated by observing EGFP fluorescence by using epifluorescent microscope (IX70, Olympus).

Table 4 indicates the percentage of the cells with EGFP fluorescence in various mammalian cell lines. The 12-well transfectable cell culture device in this inventiontransfected various mammalian cell lines with high efficiency.

### EXAMPLE 13

### Transfection with 6-Well Transfectable Cell Culture Devices Prepared with Degradable and Non-Degradable Cationic Polymers

Mammalian cells were incubated in 10-cm cell culture dishes, rinsed with phosphate-buffered saline, and treated with trypsin solution. Then, the trypsinized cells were diluted in appropriate cell culture medium with serum to prepare cell suspension. The cell density used in this example is shown in Table 4.

pEGFP-N1 plasmid was diluted in opti-MEM, and the final concentration was adjusted to 5 μg/ml. Then, 400 μl of the plasmid solution was added in each well of the 6-well transfectable cell culture device prepared as indicated in Example 10, and kept at room temperature for 25 minutes. Then, 2 ml of the cell suspension was added in the well, and incubated at 37° C. in 7.5% of CO₂. After 36 to 48-hour incubation, transfection efficiency was estimated by observing EGFP fluorescence by using epifluorescent microscope (IX70, Olympus).

Table 4 indicates the percentage of the cells with EGFP fluorescence in various mammalian cell lines. The 6-well transfectable cell culture device in this invention transfected various mammalian cell lines with high efficiency.

TABLE 4
Percentage of cells with fluorescence, and initial cell density

| | % EGFP | | | Initial Cell Density (cells/ml) | | |
|---|---|---|---|---|---|---|
| Cell Line | 6-well | 12-well | 96-well | 6-well | 12-well | 96-well |
| 293 | 80 | 80 | 80 | 2.5 × 10⁵ | 2.5 × 10⁵ | 2.5 × 10⁵ |
| 705 | 80 | 80 | 80 | 1.5 × 10⁵ | 1.5 × 10⁵ | 1.5 × 10⁵ |
| COS-7 | 70 | 70 | 70-80 | 1.5-2.0 × 10⁵ | 1.5 × 10⁵ | 1.5 × 10⁵ |
| HT-1080 | 70-80 | 70 | 70 | 0.5-1.0 × 10⁵ | 0.5 × 10⁵ | 1.0 × 10⁵ |
| HeLa | 70 | 80 | 70 | 1.0-2.0 × 10⁵ | 1.0 × 10⁵ | 0.5 × 10⁵ |
| MDCK | 50 | 60 | | 1.0 × 10⁵ | 1.5 × 10⁵ | |
| CHO-K1 | 30-40 | 50 | 50 | 1.5 × 10⁵ | 2.0 × 10⁵ | 2.0 × 10⁵ |
| DU145 | 30-40 | 40 | 30-40 | 1.5-2.0 × 10⁵ | 1.5 × 10⁵ | 1.5 × 10⁵ |
| A549 | 20-30 | 20-30 | 30-40 | 2.0 × 10⁵ | 2.0 × 10⁵ | 2.0 × 10⁵ |
| CV-1 | 20-30 | 30 | 20-30 | 1.0 × 10⁵ | 1.5 × 10⁵ | 1.5 × 10⁵ |
| HepG2 | 20 | 30 | 10-20 | 1.0-2.0 × 10⁵ | 1.5 × 10⁵ | 1.5 × 10⁵ |

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A device comprising a solid support coated with a composition comprising a transfection reagent which is not complexed to a biomolecule.

2. The device of claim 1, wherein the solid support is selected from the group consisting of polystyrene resin, epoxy resin and glass.

3. The device of claim 1, wherein the coating is on the surface of the solid support.

4. The device of claim 3, wherein the coating amount of the transfection reagent is from about 0.1 to about 100 μg/cm².

5. The device of claim 1, wherein the transfection agent is a polymer.

6. The device of claim 5, wherein the polymer is a cationic polymer.

7. The device of claim 1, wherein the transfection agent comprises a degradable cationic polymer.

8. The device of claim 7, wherein the degradable cationic polymer comprises cationic compounds or oligomers linked together by one or more degradable linkers.

9. The device of claim 7, wherein the transfection agent further comprises a non-degradable cationic polymer.

10. The device of claim 9, wherein the ratio of the non-degradable cationic polymer to the degradable cationic polymer is from 1:0.5 to 1:20 by weight.

11. The device of claim 1, wherein the transfection reagent comprises a plurality of cationic molecules and at least one degradable linker molecule connecting said cationic molecules in a branched arrangement, wherein said cationic molecules are selected from the group consisting of:
(i) a cationic compound of formula (A) or (B) or a combination thereof: 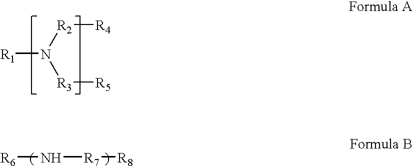
wherein R¹ is a hydrogen atom, an alkyl of 2 to 10 carbon atoms, another Formula A, or Formula B;
R² is a straight chain alkylene group of the formula: —(CH₂)ₐ— wherein a is an integer number from 2 to 10;
R³ is a straight chain alkylene group of the formula: —(C_{b}H_{2b})— wherein b is an integer number from 2 to 10;
R⁴ is a hydrogen atom, an alkyl of 2 to 10 carbon atoms, another Formula A, or Formula B;
R⁵ is a hydrogen atom, an alkyl of 2 to 10 carbon atoms, another Formula A, or Formula B;
R⁶ is a hydrogen atom, an alkyl of 2 to 10 carbon atoms, Formula A, or another Formula B;
R⁷ is a straight chain alkylene group of the formula: —(C_{c}H_{2c})— in which c is an integer number from 2 to 10; and
R⁸ is a hydrogen atom, an alkyl of 2 to 10 carbon atoms, Formula A, or another Formula B;
(ii) a cationic dendritic or branched polyamidoamine (PAMAM) with terminated primary or secondary amino groups;
(iii) a cationic polyamino acid; and
(iv) a cationic polycarbohydrate;
and wherein said degradable linker molecule is represented by the formula:
A(Z)_{d}
wherein A is a spacer molecule having at least one degradable bond, Z is a reactive residue which reacts with amino group, and d is an integer equal to or more than two and wherein A and Z are bound covalently.

12. The device of claim 8, wherein the cationic compound or oligomer is selected from the group consisting of poly(L-lysine) (PLL), polyethyleneimine (PEI), polypropyleneimine (PPI), pentaethyleneamine, N,N′-bis(2-aminoethyl)-1,3-propanediamine, N,N′-bis(2-aminopropyl)-ethylenediamine, spermine, spermidine, N-(2-aminoethyl)- 1,3-propanediamine, N-(3-aminopropyl)-1,3-propanediamine, tri(2-aminoethyl)amine, 1,4-bis(3-aminopropyl)piperazine, N-(2-aminoethyl)piperazine, dendritic polyamidoanine (PAMAM), chitosan, and poly(2-dimethylamino)ethyl methacrylate (PDMAEMA).

13. The device of claim 8, wherein the linker molecule is selected from the group consisting of di- and multi-acrylates, di- and multi-acrylamides, di- and multi-isothiocyanates, di- and multi-isocyanates, di- and multi-epoxides, di- and multi-aldehydes, di-and multi-acyl chlorides, di- and multi-sulfonyl chlorides, di- and multi-halides, di- and multi-anhydrides, di- and multi-maleimides, di- and multi-N-hydroxysuccinimide esters, di- and multi-carboxylic acids, and di-and multi-a-haloacetyl groups.

14. The device of claim 8, wherein the linker molecule is selected from the group consisting of 1,3-butanediol diacrylate, 1,4-butanediol diacrylate, 1,6-hexanediol diacrylate, 2,4-pentanediol diacrylate, 2-methyl-2,4-pentanediol diacrylate, 2,5-dimethyl-2,5-hexanediol diacrylate, poly(ethylene glycol) diacrylate, trimethylolpropane triacrylate, pentaerythritol tetraacrylate, di(trimethylolpropane) tetraacrylate, dipentaerythritol pentaacrylate, and a polyester with at least three acrylate or acrylamide side groups.

15. The device of claim 8, wherein the molecular weight of the polymer is from 500 da to 1,000,000 da.

16. The device of claim 8, wherein the molecular weight of the polymer is from 2000 da to 200,000 da.

17. The device of claim 8, wherein the molecular weight of the cationic compound or oligomer is from 50 da to 10,000 da.

18. The device of claim 8, wherein the molecular weight of the linker molecule is from 100 da to 40,000 da.

19. The device of claim 1 wherein the solid support is a dish bottom, a multi-well plate, or a continuous surface.

20. The device of claim 1, which can be stored at room temperature for at least 5 months without significant loss of transfection activity.

21. A method of cell transfection comprising:
adding a solution comprising a nucleic acid to be transfected to the device of claim 1;
adding eukaryotic cells to the device; and
incubating the cells and the nucleic acid solution to allow cell transfection.

22. The method of claim 21, wherein the incubation is for 5 min. to 3 hours.

23. The method of claim 21, wherein the incubation is for 10 min. to 90 min.

24. The method of claim 21, wherein the nucleic acid is selected from the group consisting of DNA, RNA, DNA/RNA hybrid and chemically-modified nucleic acid.

25. The method of claim 24, wherein the DNA is circular (plasmid), linear, fragment or single strand oligonucleotide (ODN).

26. The method of claim 24, wherein the RNA is single strand (ribozyme) or double strand (siRNA).

27. The method of claim 21, wherein the cell is a mammalian cell.

28. The method of claim 21, wherein at least some of the cells undergo cell division.

29. The method of claim 21, wherein the cell is a transformed or primary cell.

30. The method of claim 21, wherein the cell is a somatic or stem cell.

31. The method of claim 21, wherein the cell is a plant cell.
